(19) United States
(12) Patent Application Publication
(10) Pub. No.: US 2017/0035602 A1
(43) Pub. Date: Feb. 9, 2017

(54) DEVICE FOR COOLING AND HEATING THE NECK

(71) Applicant: SCHAWBEL TECHNOLOGIES LLC, Bedford, MA (US)

(72) Inventors: Stephen Shapiro, Burlington, MA (US); Wender Wang, Burlington, MA (US)

(21) Appl. No.: 15/107,008

(22) PCT Filed: Dec. 30, 2014
(86) PCT No.: PCT/US14/72718

§ 371 Date: Jun. 21, 2016

Related U.S. Application Data
(60) Provisional application No. 61/922,974, filed on Jan. 2, 2014.
(60) Provisional application No. 62/043,321, filed on Aug. 28, 2014.

(51) Int. Cl. A61F 7/00

(52) U.S. Cl. CPC A61F 7/007; A61F 2007/0011; A61F 2007/008; A61F 2007/0075

(57) ABSTRACT

The present invention provides devices for cooling and heating an individual's neck. Devices of the invention include two heating and cooling members that each a contoured surface for resting against a side of a user's neck and are coupled together via a bridge portion. The members each include a temperature assembly. The temperature assembly includes a heating and cooling module positioned against the contoured surface of the member. The temperature assembly may also include a heat sink, a fan, or both. The device further includes a controller for controlling the temperature assembly.

### RELATED APPLICATIONS

This application claims the benefit of and priority to U.S. Provisional No. 62/043,321, filed Aug. 28, 2014, and U.S. Provisional No. 61/922,974, filed Jan. 2, 2014. The aforementioned applications are incorporated by reference herein.

### TECHNICAL FIELD

The present application relates to a personal device for cooling and heating.

### BACKGROUND

Individuals often seek relief from cold and hot temperatures. When the weather is warm, people may utilize fans or air conditioning to cool off indoors and attempt to beat the heat outside by staying in the shade. In colder weather, people turn on the heaters, build fires and bundle up in layers of clothing, winter boots, and accessories (i.e. scarves, gloves, hats) to avoid the chill.

The above solutions, however, often leave the individual seeking further comfort in terms of heating and cooling. For example, individuals often desire a direct cooling/heating experience instead of the indirect remedies provided by air conditioners and indoor heaters.

### SUMMARY

The present invention relates to a personal device for heating and cooling that is designed to fit around the neck of an individual and adjust the temperature of an individual. Personal devices of the invention generally include two heating and cooling members or arms that are joined together via a bridge portion. The members or arms are designed to substantially rest against opposing sides of an individual's neck. For example, a first member may be contoured to fit against a right side of the neck, and the second member may be contoured to fit against the left side of the neck. The members each house a temperature assembly therein. The temperature assembly effectuates the separate heating or cooling of each member. The devices may further include a controller that is directly or indirectly coupled to the member. The controller controls the heating and cooling functions of the device.

The first and second members preferably are formed from a material that effectuates transfer of energy from the inner temperature assembly to the neck or other member part. In certain embodiments, the side of the member, which is contoured to rest against an individual's neck, includes a pad designed for transferring hot and cold temps, while maintaining comfort to the user. The pad may be formed from a phase-change material.

Each member's temperature assembly includes a heating and cooling module and may also include a heat sink, a fan, or both. In certain embodiments, the heating and cooling module is disposed within the member and positioned against an inner surface of the member that is contoured or designed to rest against a side of an individual's neck. The heat sink is also disposed within the member and is preferably positioned near the opposite side of the heating and cooling module (i.e. side of the member that opposes the neck-facing side). In certain embodiments, the heat sink rests directly against the side of the heating and cooling module that faces away from the inner surface of the member. Heat sinks suitable for use include one or more surface structures (such as cooling fins) to dissipate extra heat from an electronic source (i.e. the heating and cooling module). The heating and cooling module may further include a fan to disperse air through the heat sink. The fan may be placed within a recess created between the surface structures of the heat sink. The heat dissipated from the heat sink may exit the member from one or more outlets or vents of the members. The outlets may be located on a side of the member facing away from the individual's neck. The fan may be placed within a recess created between the surface structures of the heat sink. The positions of the heat sink, fan, and outlets are preferably such that the unwanted heat is driven away from an individual wearing the neck device.

According to certain aspects, the heating and cooling module is a Peltier system or other thermoelectric cooler. Peltier systems and thermoelectric coolers (TEC) effectuate heating and cooling by creating a heat flux between the junctions of two materials.

The temperature assemblies of the members are in electrical communication with a controller. The controller allows a user to adjust the temperature of the neck device's temperature assembly. The controller may be remotely-coupled (i.e. via a wireless device) or directly-coupled to the members. In certain embodiments, the controller is directly coupled to the members such that the controller positioned in front of an individual wearing the neck device. The controller may contain circuitry that is in electrical communication with the temperature assembly. The controller circuitry may directly control the temperature assembly. Alternatively, the temperature assembly may also include circuity that receives and carries out signals from the controller's circuitry. The circuitry of the controller and/or temperature assembly is preferably a printed circuit board (PCB). In certain embodiments, the PCB includes a processor configured to receive commands from a user and execute those commands. Execution of commands may include setting the heating and cooling module to a certain temperature.

According to aspects of the invention, a bridge portion connects the members of the neck device, and provides support to ensure the device remains around the neck while in use. In certain embodiments, the bridge portion is a strap member that rests against the back of an individual's neck. In other embodiments, the bridge portion is a junction that joins the two members or arms of the neck device and is positioned in front of an individual. The bridge portion may be directly connected to the controller or the bridge portion and the controller may be integrally formed together. In certain embodiments, the members, bridge portion, and the controller form a single, substantially rigid assembly. In other embodiments, the bridge portion is separate from the controller. When the bridge portion is separate from the controller, one or more cables can be used to connect the members to the controller. Alternatively, the controller may be wirelessly connected to the members.

The neck device may be powered by batteries or by direct charge. For battery-powered embodiments, the controller may include a compartment for receiving and coupling to one or more batteries. The compartment may further include a lid for enclosing the batteries in the housing. In other embodiments, one or more battery cells are incorporated into to a battery pack, and the battery pack may be directly clipped into the compartment of the controller. In the above embodiments, the batteries or battery pack can be removable, rechargeable, or both.

According to certain embodiments, neck devices of the invention are configured to apply direct heating or cooling to large blood vessels present on the side of one's neck. By warming or cooling the member at this location, the heating or cooling effects of the neck device may transfer through the circulatory system.

### BRIEF DESCRIPTION OF THE DRAWINGS

FIG. 1 illustrates an embodiment of a neck device of the invention.

FIG. 2 illustrates another embodiment of a neck device of the invention.

FIG. 3 illustrates a temperature assembly for use in neck devices of the invention.

FIG. 4 illustrates positioning of a fan and a vent for dissipating undesirable or excess heat from the heating and cooling module.

FIG. 5 illustrates the neck device of FIG. 2 in the open configuration.

FIG. 6A illustrates a heat sink according to certain embodiments.

FIG. 6B illustrates a fan for use with the heat sink of FIG. 6A.

FIGS. 7A and 7B illustrate another heat sink according to certain embodiments.

### DETAILED DESCRIPTION

The present invention provides personal devices for heating and cooling. Device of the invention include two members that are connected via a bridge. The members are contoured to fit against the side of one's neck, while the bridge provides support to maintain the positioning of the members on the neck of an individual. Each member includes its own temperature assembly, which generates and emits heating or cooling temperatures. By heating and cooling one's neck, blood passing through large blood vessels located near the neck surface is also heated or cooled. The effects of applying hot/cold temperatures to blood flowing through the neck may then be transferred throughout the circulatory, thereby passing the heating/cooling effects throughout one's member. While devices of the invention are particularly useful for cooling and heating the neck, the devices may be used to heat or cool any other body part (including legs, arms, etc.).

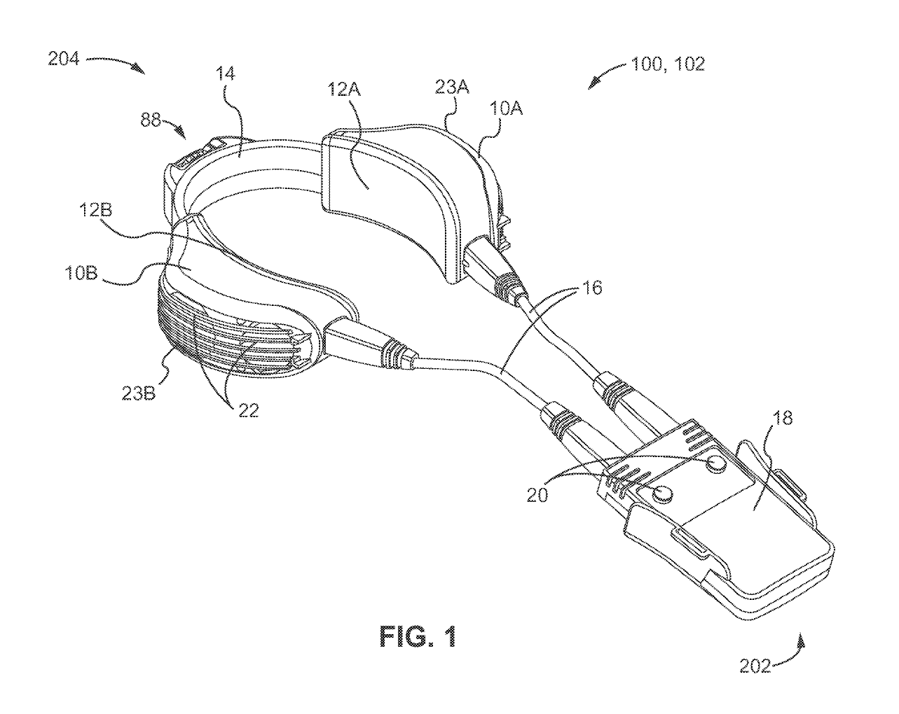

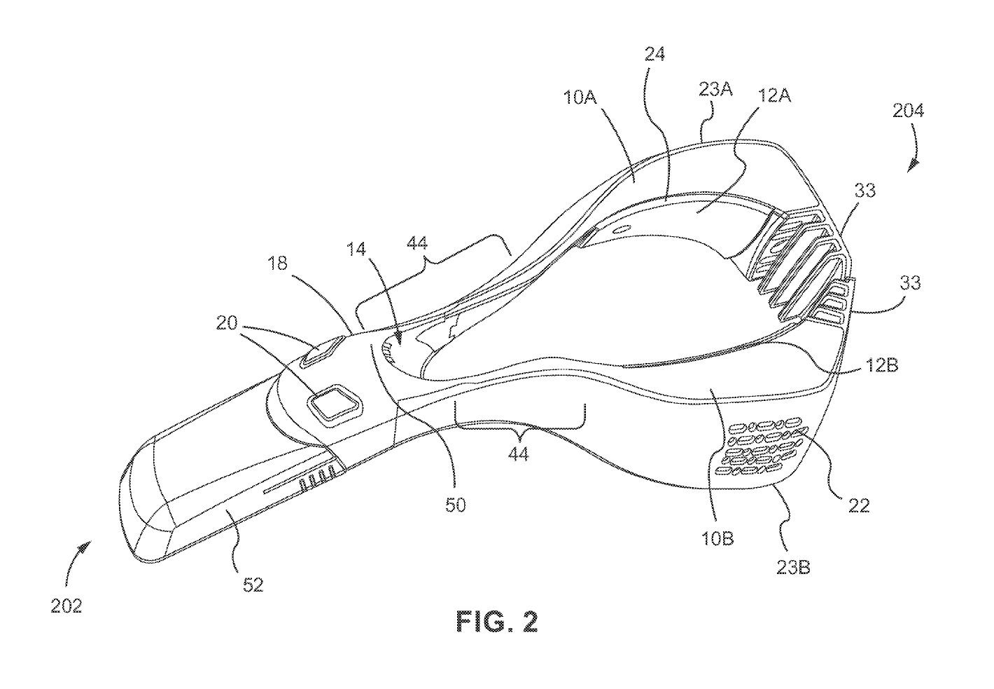

FIGS. 1 and 2 illustrate different designs 102, 104 of the heating and cooling devices 100 of the invention. The varying designs 102, 104 of heating and cooling devices 100 of FIGS. 1 and 2 are separately described below. In the figures, reference character 202 shows a proximal location relative to the device and reference character 204 shows a distal location relative to the device.

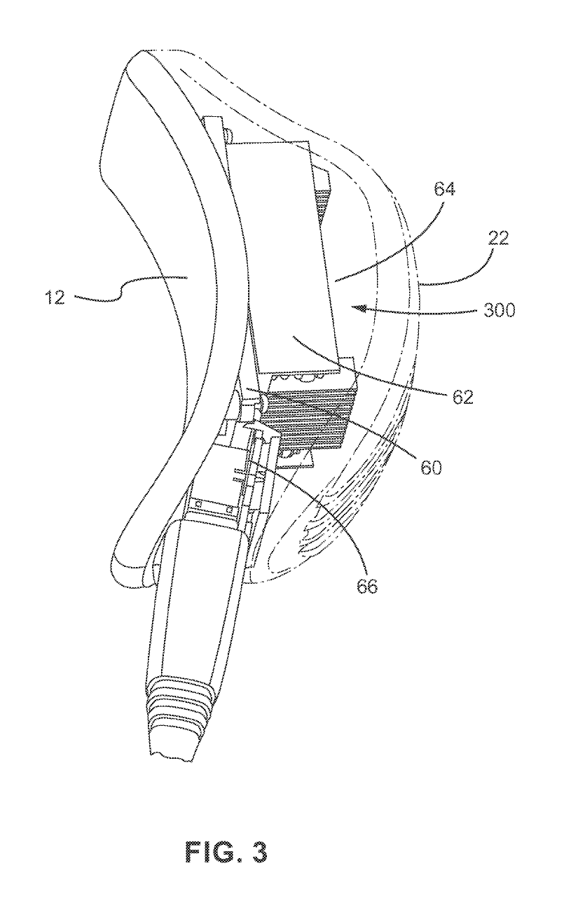

FIG. 1 illustrates a neck cooling/heating device 102. As shown in FIG. 1, the neck cooling device 102 includes two heating/cooling members (or arms) 10A, 10B, a controller 18, and a bridge portion 10. The neck device 102 is designed to fit around the neck of an individual such that the two members 10A, 10B fit adjacent to the sides of an individual's neck. In certain embodiments, the members 10A, 10B includes outer surfaces 12A, 12B that are contoured to rest against an individual's neck. The contouring provides comfort and increases heat transfer surface area, i.e. area in which heating/cooling members 10A, 10B are in direct contact with the neck. The first and second members 10A, 10B preferably are formed from a material that is designed to effectuate transfer of heating and cooling from an inner temperature assembly (described hereinafter; see FIG. 3) to the neck. In certain embodiments, a pad is coupled to the surfaces to increase heat transfer and comfort (see pad 24 in FIG. 2). The pad may be formed from a phase-changing material (described hereinafter).

The sides 23A, 23B of the members 10A, 10B, which oppose the neck-facing surfaces 12A, 12B, include one or more vent openings 22. The vent openings 22 allow extra or unwanted heat to be dissipated from the members 10A, 10B, e.g., when the device is in cooling mode. The members 10A, 10B are coupled together via a bridge member 14, which rests against the back of an individual's neck who is wearing the neck device. As shown, the bridge member 14 is a strap member spanning from the distal ends of the members 10A, 10B. The bridge member 14 is preferably semi-rigid to maintain positioning of the device 102 around the neck when in use, while allowing a user to easily place the device 102 on the neck and remove the device 102 from the neck. The bridge member 14 may include a toggle 88 to adjust the length or flexibility of the bridge member 14.

A controller 18 may be coupled to the members 10A, 10B either directly or remotely (e.g., wireless connection). The controller 18 controls the temperature assembly (described hereinafter) disposed within each member 10A, 10B. For example, the controller allows one to turn the temperature assembly on/off and set a heating/cooling setting. As shown, the members 10A, 10B are directly coupled to the controller 18 via cords 16. The cords 16 transfer electrical signals and energy from the controller 18 to the members 10A, 10B. The energy may be transferred from one or more batteries coupled to the controller 18. The controller 18 includes user command buttons 20 for sending signals through the controller to the heating/cooling members 10A, 10B. Alternatively, the controller 18 may be wirelessly connected to the members 10A, 10B. For example, the controller 18 may be a remote module that wirelessly connects to the temperature assembly.

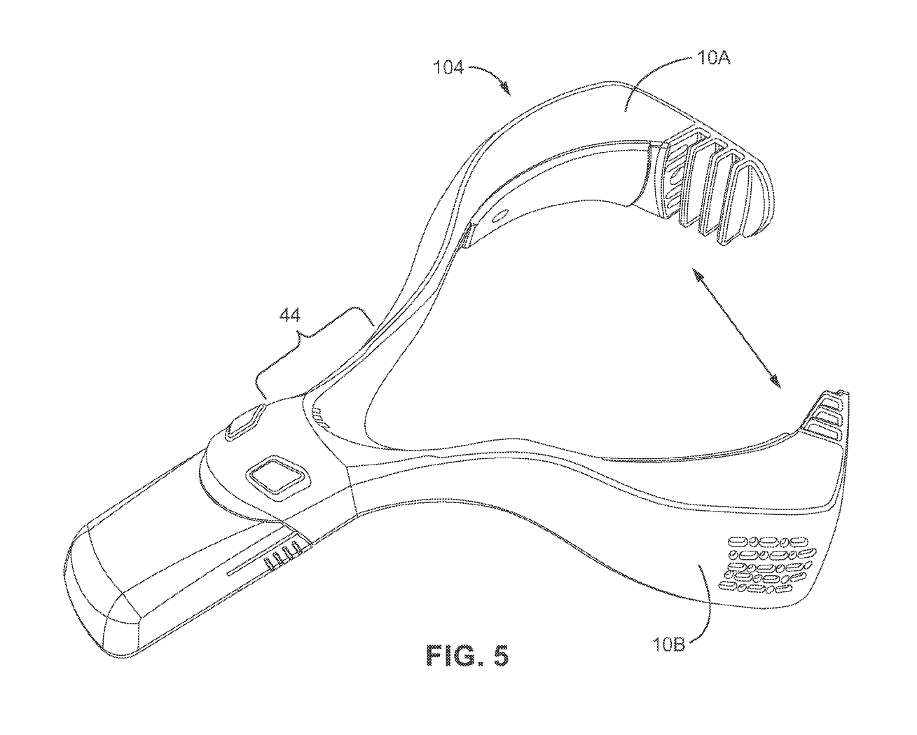

FIG. 2 illustrates an additional design 104 of a neck heating and cooling device 100 according to certain aspects of the invention. As shown in FIG. 2, the neck device 104 likewise has two members (or arms) 10A, 10B configured to rest against a user's neck, a bridge portion 14, and a controller 18. The neck device of FIG. 2 differs, however, in the configuration of these elements. In device 102, the members 10A, 10B, bridge portion 14, and controller 18 are joined/formed together to form a single unit that is substantially rigid, but for movement of the arms 10A, 10B relative to each other. As shown in FIG. 2, members 10A, 10B are coupled to together at a junction. The junction is the bridge section 14 of the device 104 shown in FIG. 2. According to certain embodiments, the device includes moveable portions 44 that are included within the bridge section 14 or coupled to the bridge section 14. The movable portions 44 allow the members 10A, 10B to move apart from each other. This movement allows a user to place the device around his/her neck. For example, the moveable portion 44 allows a user to open the spread the members 10A, 10B. When spread, a user can place the device onto the user's neck such that the bridge portion 14 and controller 18 are positioned against the chest of the user (i.e. in front of the user). The moveable portion 44 may include a hinge, flexible material, or both. For example, the moveable portion 44 may include two or more components that are hinged to each other to allow movement of the first member 10A relative to the second member 10B. In some embodiments, the hinged components may be covered by a flexible material. In other embodiments, the moveable portion 44 is formed from a flexible shape-memory polymer that allows movement of the arms upon application of pressure, but returns to its shape once pressure is released. FIG. 5 illustrates the device 104 with the arms 10A, 10B separated by a distance, as allowed by the moveable portion 44.

The bridge section 14 of the device 102, shown in FIG. 2, is directly coupled to or integrally formed with the controller 18. As shown, the bridge member 14 spans from the proximal ends of the members 10A, 10B. The controller 18 and bridge section 14 are positioned such that they rest on an individual chest, when the device is being worn by a user. Alternatively, the controller 18 may be separated from the bridge section 14, and wirelessly coupled to the device 102. In such instances, the controller 18 is a remote control used to wireless control the temperature assembly disposed within the members 10A, 10B. In certain embodiments, the controller 18 may include a compartment or housing configured to receive a removeable battery pack 52. The battery pack 52 is a container for holding two or more rechargeable batteries therein. The removable battery pack may clip into the compartment during use, and removed from the compartment for recharging. In other embodiments, the batteries may be placed in the compartment, and a lid may be used to enclose the batteries therein.

According to certain embodiments, the members 10A, 10B also include flanges or extension members 33 extending from the distal ends of the members 10A, 10B. The flanges 33 rest substantially against a back of a user's neck, when wearing the neck cooling device, and prevent the neck cooling device from slipping or falling off the user's neck. The flanges 33 may be made from the same or different material as the members 10A, 10B. Preferably, the flanges 33 are formed from a more flexible material than a material forming the members 10A, 10B. As shown, the flanges 33 include several cutouts to increase flexibility and provide additional comfort to the wearer.

As discussed and shown in FIGS. 1 and 2, both neck devices 100 generally include heating/cooling members 10A, 10B, a bridge portion 14, and a controller 18. These features and additional features of the neck device 100 are described in more detail hereinafter.

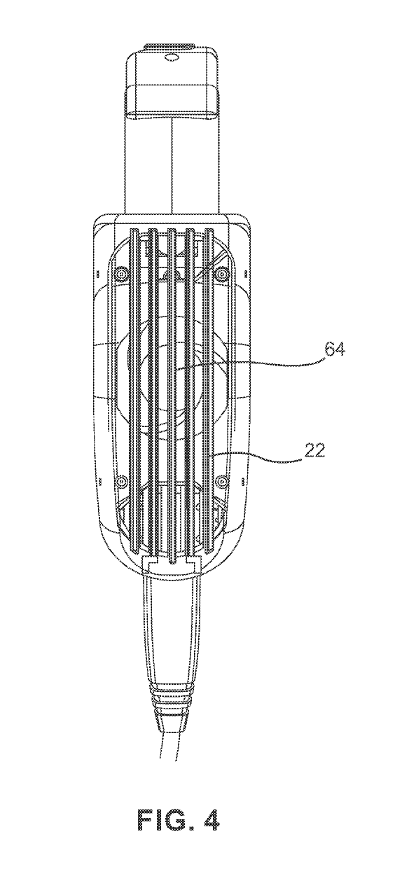

The members 10A, 10B each contain or house a temperature assembly 300 that effectuates the separate heating or cooling of each member. The temperature assembly 300 housed within a member 10A, 10B is best shown in FIG. 3. The temperature assembly 300 includes a heating and cooling module 60. Alternatively, the temperature assembly 300 may include just a heating module or just a cooling module. The temperature assembly 300 may also include a heat sink 62, a fan 64, or both. The heating and cooling module 60 is positioned adjacent to outer surface 12A, 12B of the member 10A, 10B that is contoured or designed to rest against the side of an individual's neck. The heat sink 62 is preferably placed directly on the back side of the heating and cooling module 60, i.e. the side that opposes the side adjacent to the inner surface 12. Heat sinks are generally known, and include one or more surface structures (such as cooling fins) to dissipate extra heat from an electronic source (i.e. the heating and cooling module). A fan 64 may be used to dissipate heat through the heat sink 62. The dissipated heat may then be transferred by the fan through one or more vents 22. In certain embodiments, the positioning of the heat sink 62, fan 64, and vents 22 are such that the unwanted heat is driven away from an individual wearing the neck device 100. FIG. 4 illustrates a fan 64 centrally located within the heat sink 62 of a member 10. The centrally-located fan 64 blows air from the vents 22, which are located on a side of the member facing away from the individual's neck.

According to certain aspects, the heating and cooling module 60 is a Peltier system or other thermoelectric cooler. Peltier systems and thermoelectric coolers (TEC) effectuate heating and cooling by creating a heat flux between the junctions of two different materials. Generally, a thermoelectric cooling element provides localized heating and cooling of the members 10A, 10B through use of the Peltier effect to create a heat flux. Thermoelectric cooling elements include a first and a second substrate separated by two or more semi-conductors wires (such as alternating p-types and c-types). When the semi-conductors wires are connected to each other by a positive and negative power source, heat is transferred from the first substrate, which effectuates cooling of the first substrate, to the second substrate, which effectuates heating of the second substrate. The semi-conductor wires of the temperature assembly may be coupled to the positive and negative power source from the controller 18. The heated substrate is typically associated with a heat sink (such as heat sink 62) that acts to dissipate the heat away from the second substrate. The combination of the thermoelectric cooling element with a heat sink ideally cools/heats the members 10A, 10B while safely dissipating unwanted heat through the vents 22.

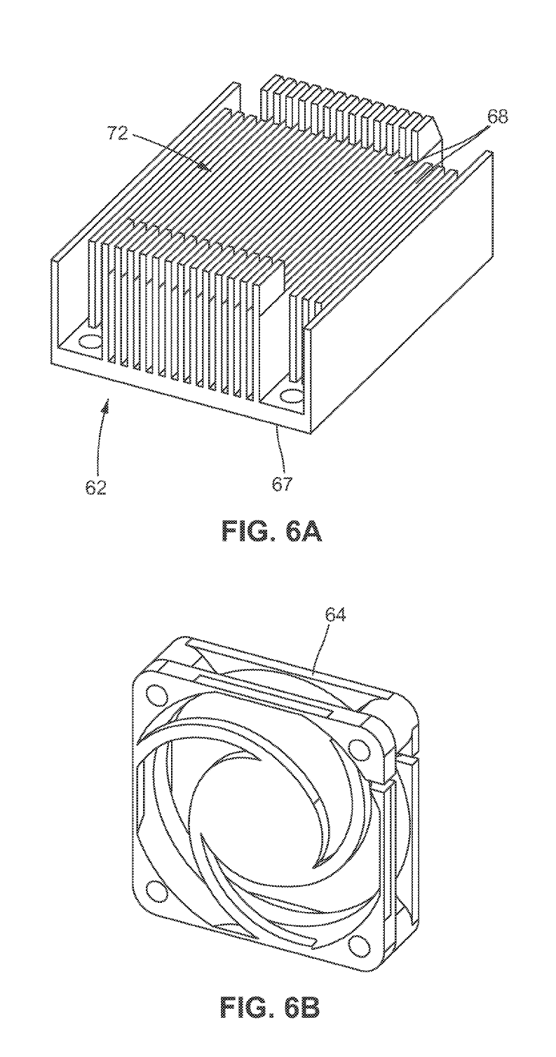

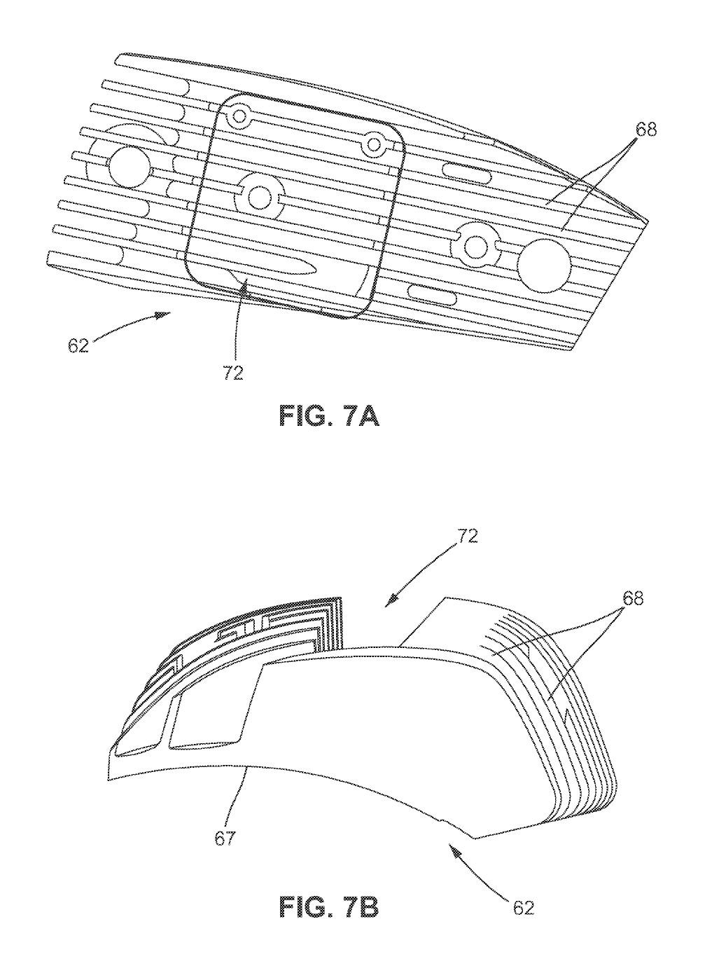

FIG. 6A illustrates a heat sink 62 according to certain embodiments. The heat sink 62 includes a plurality of fins 68 and a recess 72 defined within the fins. The fins 68 form a top surface of the heat sink and increase the surface area used to effectuate heat transfer. The recess is configured to receive the fan 64 for driving heat from the heating and cooling module 60 through the heat sink 62 and expelling heated air towards the vents 22 of the members 10A, 10B. FIG. 6B illustrates the fan 64 (blades not shown) for placement in the recess 72. Referring back to FIG. 6A, the heat sink 62 also includes a flat bottom surface 67. The flat surface 67 rests directly against the heating and cooling module 60, which are typically flat. Alternatively, the heat sink 62 may have a curved bottom surface 67 (as shown in FIGS. 7A and 7B). In such instances, the curved bottom surface 67 may include a recess for accommodating a flat heating and cooling module 60 therein. Alternatively, the curved surface 67 may be configured to rest against a similarly curved heating and cooling module 60. The heat sink 62 of FIGS. 7A and 7B also has a recess 72 defined within cooling fins to receive a fan 64.

The temperature assembly 300 may be directly coupled to the controller 18 or wirelessly coupled to the controller 18. As shown in FIGS. 3 and 4, the temperature assembly 300 is directly coupled to the controller via an electrical junction 66.

The controller 18 is configured to send signals to the temperature assembly 300 of the members 10A, 10B to effectuate heating and cooling of the device 100. In certain embodiments, the controller 18 allows a user to adjust the temperature of the neck device's temperature assembly 300 via user controls 20. Although shown directly-coupled, the controller 18 may be remotely-coupled (i.e. via a wireless device) or directly-coupled to the members. The controller 18 may contain circuitry that is in electrical communication with the temperature assembly 300. The controller circuitry may directly control the temperature assembly. Alternatively, the temperature assembly may also include circuity that receives signals and carries out commands from the controller's circuitry. The circuitry of the controller and/or temperature assembly is preferably a printed circuit board (PCB). In certain embodiments, the PCB includes a processor configured to receive commands from a user and execute those commands. Execution of commands may include turning the heating and cooling module on/off or setting the heating and cooling module to a certain temperature. The processor may also be configured to automatically generate commands. For example, the processor may include logic for a feedback loop that maintains the temperature of the heating/cooling device. In such instance, the processor may include means of determining the temperature of either one of the members 10A, 10B, and adjusting the temperature to maintain a constant temperature (e.g. at a set temperature).

The controller 18 may send the same instructions to the temperatures assembly 300 of each member 10A, 10B, or the controller 18 may send different instructions to the temperature assembly of each member 10A, 10B. For example, the controller 18 may cause both members 10A, 10B to emit heat of a certain temperature. Alternatively, the controller 18 may cause member 10A to emit a first heat, and cause member 10B to emit a second heat. In certain embodiments, the controller 18 may be used to generate cyclic temperature changes (e.g. on at a certain temperature for a period of time and off for a period of time).

The temperature assembly 300 may have several temperature settings, ranging in cold to hot temperatures. The temperature may range from for example, −5° C. to 50° C. The cold settings may range from low (cool), medium, and high (coldest). The hot settings may range from low (warm), medium, and high (warmest).

The neck device 100 may be powered by batteries or by direct charge. The batteries are in electrical communication with the PCB (of the controller, temperature assembly, or both) and the temperature assembly. In certain embodiments, the PCB directs transfer of energy from the battery to the heating and cooling module of the temperature assembly. In addition to powering the heating and cooling module, the battery may be used to power the fan of the heat sink. For direct charge embodiments, the neck device may include a port for plugging an external charger directly into the neck device. The port may be, for example, a USB port. For battery-powered embodiments, the controller may be designed to couple to one or more batteries. For example, the controller may define a compartment that receives one or more batteries. The battery may be the battery itself (i.e. one or more battery cells) or a battery pack, which is a member that encloses one or more battery cells. In certain embodiments, the battery pack may be directly clipped onto the controller. FIG. 2 illustrates a removable battery pack 52 that can be directly clipped onto the controller compartment. The removable battery pack may clip into the compartment during use, and removed from the compartment for recharging. In other embodiments, the housing may include a compartment configured to receive one or more batteries. The batteries may be placed in the housing, and a lid may be used to enclose the batteries therein. The battery can be inserted and removed from the controller at the user's convenience.

Any suitable battery may be used for the battery or battery cell. The batteries can be removable, rechargeable, or both. Types of batteries include, for example, nickel cadmium, nickel-metal hydride, lead acid, lithium ion, lithium ion polymer batteries. The battery chosen ideally holds charge for more than 2, 3, 4 or 5 hours, and is rechargeable.

Suitable materials for the neck devices 100 of the invention are described hereinafter. The members, controller, and bridge section may be formed from the same material or different materials. Suitable materials may include metal, plastics, polymers, or polymeric blends. The material chosen may be thermally conductive, thermally insulative, lightweight, and/or water-resistant. In certain embodiments, the members are formed from a thermally conductive material on the side resting against the neck and a thermally insulative material on the side facing away from the neck. Suitable polymeric materials include Polyethylene terephthalate (PET), Polyethylene (PE), High-density polyethylene (HDPE), Polyvinyl chloride (PVC), Polyvinylidene chloride (PVDC), Low-density polyethylene (LDPE), Polypropylene (PP), Polystyrene (PS), High impact polystyrene (HIPS), and combinations thereof. Suitable metals include steel, aluminum, copper, etc.

In certain embodiments, personal devices 100 (FIG. 1 or 2) may include a pad 24 coupled to the outer surfaces 12A, 12B that rest against a user's neck. The pad 24 may be used to provide comfort to a wearer of the device 100. In addition, the pad 24 may be used to enhance hot/cold transfer from the device 100 to the user. In such instances, the pad 24 may be formed from a phase-change material. In general, a phase change material can be any substance (or any mixture of substances) that has the capability of absorbing or releasing thermal energy to regulate, reduce, or eliminate heat flow within a temperature stabilizing range. The temperature stabilizing range can include a particular transition temperature or a particular range of transition temperatures. When used in conjunction with device 100, the PCM(s) transition between storing heat and releasing heat in response to energy outputted by the temperature assembly.

A phase-change material (PCM) is a substance that melts and solidifies at a certain temperature. Heat is absorbed or released when the material changes from solid to liquid and vice versa; thus, PCMs are classified as latent heat storage (LHS) units. PCMs latent heat storage can be achieved through solidsolid, solidliquid, solidgas and liquidgas phase change. Preferably, the PCM material used in the heated packs transitions from solid to liquid phase. Initially, the solidliquid PCMs behave like sensible heat storage (SHS) materials; their temperature rises as they absorb heat. When PCMs reach the temperature at which they change phase (their melting temperature) they absorb large amounts of heat at an almost constant temperature. The PCM continues to absorb heat without a significant rise in temperature until all the material is transformed to the liquid phase. When the ambient temperature around a liquid material falls, the PCM solidifies, releasing its stored latent heat. A large number of PCMs are available in any required temperature range from 5 up to 190° C., in which the human comfort range is between 20-30° C. They may store 5 to 14 times more heat per unit volume than conventional storage materials such as water, masonry or rock.

PCM materials may be formed from organic substances, inorganic substances or polymeric substances. Examples of organic or inorganic phase change materials include hydrocarbons (e.g., straight-chain alkanes or paraffinic hydrocarbons, branched-chain alkanes, unsaturated hydrocarbons, halogenated hydrocarbons, and alicyclic hydrocarbons), hydrated salts (e.g., calcium chloride hexahydrate, calcium bromide hexahydrate, magnesium nitrate hexahydrate, lithium nitrate trihydrate, potassium fluoride tetrahydrate, ammonium alum, magnesium chloride hexahydrate, sodium carbonate decahydrate, disodium phosphate dodecahydrate, sodium sulfate decahydrate, and sodium acetate trihydrate), waxes, oils, water, fatty acids, fatty acid esters, dibasic acids, dibasic esters, 1-halides, primary alcohols, secondary alcohols, tertiary alcohols, aromatic compounds, clathrates, semi-clathrates, gas clathrates, anhydrides (e.g., stearic anhydride), ethylene carbonate, polyhydric alcohols (e.g., 2,2-dimethyl-1,3-propanediol, 2-hydroxymethyl-2-methyl-1,3-propanediol, ethylene glycol, polyethylene glycol, pentaerythritol, dipentaerythritol, pentaglycerine, tetramethylol ethane, neopentyl glycol, tetramethylol propane, 2-amino-2-methyl-1,3-propanediol, monoaminopentaerythritol, diaminopentaerythritol, and tris(hydroxymethyl)acetic acid), polymers (e.g., polyethylene, polyethylene glycol, polyethylene oxide, polypropylene, polypropylene glycol, polytetramethylene glycol, polypropylene malonate, polyneopentyl glycol sebacate, polypentane glutarate, polyvinyl myristate, polyvinyl stearate, polyvinyl laurate, polyhexadecyl methacrylate, polyoctadecyl methacrylate, polyesters produced by polycondensation of glycols (or their derivatives) with diacids (or their derivatives), and copolymers, such as polyacrylate or poly(meth)acrylate with alkyl hydrocarbon side chain or with polyethylene glycol side chain and copolymers including polyethylene, polyethylene glycol, polyethylene oxide, polypropylene, polypropylene glycol, or polytetramethylene glycol), metals, and mixtures thereof.

Polymeric phase change materials can be formed by polymerizing octadecyl methacrylate, which can be formed by esterification of octadecyl alcohol with methacrylic acid. Also, polymeric phase change materials can be formed by polymerizing a polymer (or a mixture of polymers). For example, poly-(polyethylene glycol) methacrylate, poly-(polyethylene glycol) acrylate, poly-(polytetramethylene glycol) methacrylate, and poly-(polytetramethylene glycol) acrylate can be formed by polymerizing polyethylene glycol methacrylate, polyethylene glycol acrylate, polytetramethylene glycol methacrylate, and polytetramethylene glycol acrylate, respectively. In this example, the monomer units can be formed by esterification of polyethylene glycol (or polytetramethylene glycol) with methacrylic acid (or acrylic acid). It is contemplated that polyglycols can be esterified with allyl alcohol or trans-esterified with vinyl acetate to form polyglycol vinyl ethers, which in turn can be polymerized to form poly-(polyglycol) vinyl ethers. In a similar manner, it is contemplated that polymeric phase change materials can be formed from homologues of polyglycols, such as, for example, ester or ether endcapped polyethylene glycols and polytetramethylene glycols.

Due to the transitioning nature of PCMs (solid-liquid), it is desirable to contain the PCM materials. The phase-change material may be encapsulated (e.g. in a microcapsule) or may be contained within a fiber. Microcapsules can be formed as shells enclosing a phase change material, and can include individual microcapsules formed in various regular or irregular shapes (e.g., spherical, spheroidal, ellipsoidal, and so forth) and sizes. Microcapsules containing PCM materials can be used in a variety of manners. For example, PCM microcapsules may be used to coat a polymeric or fabric layer. Alternatively, PCM microcapsules can be dispersed throughout a polymeric or fabric layer. In other embodiments, PCM can be directly incorporated into a fibers used to make fabrics. The PCM, may be located, within the core of a cellulosic fiber. In certain embodiments, fibers with PCMs incorporated therein include acrylic, viscose, and polyester fibers.

The type of PCM material chosen may be dependent on the desired temperature range of the device 100. A transition temperature of a phase change material typically correlates with a desired temperature or a desired range of temperatures that can be maintained by the phase change material. For example, a phase change material may be selected because it has a transition temperature near the desired energy outputs (e.g. low, medium, high) of the heated pack 100. In some instances, a phase change material can have a transition temperature in the range of about 5° C. to about 125° C., such as from about 0° C. to about 100° C., from about 0° C. to about 50° C., from about 15° C. to about 45° C., from about 22° C. to about 40° C., or from about 22° C. to about 28° C.

PCMs are described in more detail in U.S. Pat. Nos. U.S. Pat. No. 6,855,422; U.S. Pat. No. 7,241,497; U.S. Pat. No. 7,160,612; U.S. Pat. No. 7,666,502; U.S. Pat. No. 7,666,500; U.S. Pat. No. 6,793,856; U.S. Pat. No. 7,563,398; U.S. Pat. No. 7,135,424; U.S. Pat. No. 7,244,497; U.S. Pat. No. 7,579,078; and U.S. Pat. No. 7,790,283. Also, the following references discuss phase-changing materials in more detail: Kenisarin, M; Mahkamov, K (2007). “Solar energy storage using phase change materials”. Renewable and Sustainable Energy Reviews 11(9): 1913-1965; Sharma, Atul; Tyagi, V. V.; and Chen, C. R.; Buddhi, D. (2009). “Review on thermal energy storage with phase change materials and applications”. Renewable and Sustainable Energy Reviews 13 (2): 318-345.

As discussed above, the controller 18 may be remotely connected to the temperature assembly of the members 10A, 10B. Remote control technology is generally known, and relies on sending a signal, such as light, Bluetooth (i.e. ultra-high frequency waves), and radiofrequency, to operate a device or circuit. Dominant remote control technologies rely on either infrared or radiofrequency transmissions. A radiofrequency remote transmits radio waves that correspond to the binary command for the button you're pushing. As applicable to the device 100, the command may include, for example, high heat, low heat, medium heat, high cool, low cool, medium cool, on, or off. A radio receiver on the members (e.g. circuit of temperature assembly 300) receives the signal and decodes it. The receiver then transmits the decoded signal to the circuitry, and the circuitry executes the command. The above-described concepts for radiofrequency remote controls are applicable for light and Bluetooth remote controls.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A device for heating and cooling an individual's neck, the device comprising:
a first member and a second member, the members each comprising a temperature assembly disposed therein and an outer surface that is contoured to rest against a side of the neck, the temperature assembly comprising:
a heating and cooling module positioned against the contoured outer surface;
a heat sink operably associated with the heating and cooling module; and
a fan operably associated with the heat sink;
a controller for controlling the temperature assembly; and
a bridge portion connecting the first and second members.

2. The device of claim 1, wherein the heating and cooling module comprises a thermoelectric cooler.

3. The device of claim 1, wherein the contoured surface comprises a pad.

4. The device of claim 1, wherein the controller comprises a printed circuit board that is in electrical communication with the temperature assembly.

5. The device of claim 1, wherein the device further comprises a battery in electrical communication with printed circuit board and the temperature assembly.

6. The device of claim 1, wherein the bridge portion is directly coupled to the controller.

7. The device of claim 6, wherein the bridge portion, controller, and members form a single unit.

8. The device of claim 1, wherein the bridge portion is not directly connected to the controller.

9. The device of claim 8, wherein one or more cables connect the first and second members to the controller.

10. The device of claim 1, wherein the controller comprises a compartment for receiving a battery.

11. The device of claim 1, further comprising a battery pack that is configured to attach to and detach from the controller.

12. A device for heating and cooling an individual's neck, the device comprising
a first member and a second member, the members each comprising a distal end, a proximal end, a temperature assembly disposed therein, and an outer surface that is contoured to rest against a side of the neck, the temperature assembly comprising:
a heating and cooling module positioned against the contoured outer surface;
a heat sink operably associated with the heating and cooling module; and
a fan operably associated with the heat sink; and
a bridge portion coupled to the distal ends of the members and connecting the members to each other, the bridge portion configured to rest against the back of the individual's neck.

13. The device of claim 12, wherein the device further comprises a controller operably coupled to the proximal ends of the members.

14. The device of claim 13, wherein cables couple the controller to the proximal ends of the members.

15. The device of claim 13, wherein the controller is wirelessly coupled to the members.

16. The device of claim 12, wherein the bridge portion comprises a toggle for adjusting a length of the bridge portion.

17. A device for heating and cooling an individual's neck, the device comprising
a first arm and a second arm, each arm comprising:
a distal end and a proximal end;
an outer surface that is contoured to rest against a side of the neck;
a heating and cooling module disposed within the arm and positioned against the contoured outer surface; and
an extension member that extends from the distal end of the arm and is configured to rest against a back of the neck; and
a bridge portion coupled to the proximal ends of the arms and connecting the arms to each other, the bridge portion configured to rest against the chest of the individual.

18. The device of claim 17, wherein the bridge portion is directly coupled to a connector.

19. The device of claim 18, wherein the connector comprises a compartment for receiving a battery.

20. The device of claim 19, wherein the battery is a battery pack.
